(19) United States
(12) Patent Application Publication
(10) Pub. No.: US 2026/0083927 A1
(43) Pub. Date: Mar. 26, 2026

(54) CONTROL UNIT FOR A MEDICAL VENTILATOR

(71) Applicant: Loewenstein Medical Technology S.A., Luxembourg (LU)

(72) Inventors: Peter KREMEIER, Karlsruhe, DE (US); Gerardo TUSMAN, Mar del Plata, AR (US)

(21) Appl. No.: 19/331,097

(22) Filed: Sep. 17, 2025

(30) Foreign Application Priority Data
Sep. 23, 2024 (DE) 10 2024 127 439.5

(51) Int. Cl. A61M 16/00; G16H 40/63

(52) U.S. Cl. CPC A61M 16/024; G16H 40/63; A61M 2016/0027; A61M 2205/3306; A61M 2205/3334; A61M 2230/432; A61M 2230/46

(57) ABSTRACT

A medical ventilator comprises a respiratory air connection for connecting a patient's respiratory apparatus, allowing the patient to be ventilated with breathable air. In addition, the ventilator comprises an actuator device for providing a respiratory air flow at the respiratory air connection. A control unit is configured to carry out a method as set forth in the claims when the respiratory apparatus is connected to the respiratory air connection.

### CROSS-REFERENCE TO RELATED APPLICATIONS

The present application claims priority under 35 U.S.C. § 119 of German Patent Application No. 10 2024 127 439.5, filed Sep. 23, 2024, the entire disclosure of which is expressly incorporated by reference herein.

### BACKGROUND OF THE INVENTION

### 1. Field of the Invention

The invention relates to a control unit for a medical ventilator. Furthermore, the invention relates to a computer program executable by the control unit, a corresponding computer-readable medium and a medical ventilator having such a control unit.

### 2. Discussion of Background Information

In artificial ventilation, the so-called dead space volume plays an important role. This can generally be understood as the proportion of the tidal volume that remains within an anatomical and instrumental dead space (also called dead space ventilation) during each breath and thus does not participate in the alveolar gas exchange. During each exhalation phase the dead space is filled with carbon dioxide, which is then inhaled again during the following inhalation phase. Due to the dead space, an undesirable level of carbon dioxide may be retained in the body, a situation which in some cases can cause hypercapnia or respiratory acidosis. For example, in ARDS and COPD patients (ARDS=acute respiratory distress syndrome; COPD=chronic obstructive pulmonary disease), the dead space ventilation can amount to at least half of the total ventilation per unit time. This can significantly reduce the efficiency of ventilation. In principle, a reduction in dead space ventilation, i.e. an improvement in carbon dioxide elimination, can have a protective effect on the lungs, because this allows a lower tidal volume and/or a lower airway pressure to be used.

The dead space can be reduced, for example, by reducing the number and/or volume of the components connecting the respiratory apparatus to the medical ventilator. However, such a reduction in dead space cannot be easily implemented in practice. In addition, it is possible to increase carbon dioxide elimination by means of a suitable extracorporeal method such as extracorporeal membrane oxygenation (ECMO) or extracorporeal carbon dioxide elimination (ECCO₂R). However, these methods are highly invasive and technically very demanding.

In view of the foregoing it would be advantageous to have available a control unit that enables the carbon dioxide elimination during ventilation of a patient to be improved in a simple and stress-free manner. It also would be advantageous to have available a corresponding computer program, a corresponding computer-readable medium and a corresponding medical ventilator.

### SUMMARY OF THE INVENTION

In a first aspect, the invention provides a control unit for a medical ventilator. The ventilator comprises a respiratory air connection for connecting a patient's respiratory apparatus, allowing the patient to be ventilated with breathable air. In addition, the ventilator comprises an actuator device for providing a respiratory air flow to the respiratory air connection. The control unit is configured to carry out the following method when the respiratory apparatus is connected to the respiratory air connection: generating a control signal for controlling the actuator device, so that at least one actual quantity relevant to the ventilation, which comprises a pressure and/or a volume of the respiratory air, follows a target curve between a lower limit and an upper limit during each breath, wherein in an inhalation phase in which the patient should inhale, the target curve rises from the lower limit to the upper limit and in an exhalation phase in which the patient should breathe out, it falls from the upper limit to the lower limit; (during the ventilation and/or when the actuator device is controlled:) switching the lower limit between a first setpoint and a second setpoint, which is smaller in magnitude than the first setpoint, according to a cyclically repeating breathing sequence, wherein the breathing sequence in each repetition comprises one or more first breaths, in which the at least one actual quantity is expected to fall to the first setpoint in the (respective) exhalation phase, and one or more second breaths, in which the at least one actual quantity is expected to fall to the second setpoint in the (respective) exhalation phase, wherein each second breath or each sequence of immediately consecutive second breaths is immediately preceded by a first breath or a sequence of immediately consecutive first breaths.

In this way, a targeted periodic fluctuation of the functional residual capacity, FRC for short, can be effected over one or more breaths. This in turn can have a beneficial effect on the carbon dioxide elimination. In particular, such control of the respective ventilation-relevant quantity(ies) allows a significant increase in the average carbon dioxide elimination without necessarily increasing the volume exhaled by the patient. Such an increase in the tidal volume should be avoided in the interest of making the ventilation process as stress-free as possible.

In any case, the lower limit should be chosen in such a way that a lung collapse during exhalation is avoided. At the same time, the difference between the first setpoint and the second setpoint should be chosen large enough to achieve a therapeutically effective increase in the carbon dioxide elimination.

For example, the lower limit may comprise a lower target pressure to which the pressure of the respiratory air should drop in each exhalation phase. Similarly, the upper limit may comprise an upper target pressure. The lower target pressure can also be referred to as the positive end-expiratory pressure, PEEP for short. The lower target pressure can be understood to be a pressure smaller in magnitude than the upper target pressure.

In addition or alternatively, the lower limit may comprise a lower target volume to which the volume of the respiratory air should fall in each exhalation phase. Similarly, the upper limit may comprise an upper target volume. The lower target volume can be understood to be a volume smaller in magnitude than the upper target volume.

It is possible that the ventilator also comprises a sensor device for generating measurement data regarding the ventilation (see also below). The sensor device may comprise, for example, a pressure sensor for detecting the pressure of the respiratory air and/or a flow sensor for detecting a volume flow of the respiratory air. In this case, the method can further comprise: receiving the measurement data, wherein the measurement data comprises one or more values for the actual quantity (ies) (acquired during ventilation). Accordingly, it is possible that the control signal is generated using the measurement data, in particular in such a way that the respective actual quantity, more precisely its curve, approximates to the target curve by means of appropriate control of the actuators. The measurement data can additionally or alternatively be used to adjust the target curve (for example, the first setpoint and/or the second setpoint) and/or the breathing sequence.

The respiratory air pressure can be, in a broader sense, a pressure of the respiratory air in the patient's lungs. In a similar manner, the volume of respiratory air in the broader sense can be an air-filled volume of the lungs.

It is possible that at least one repetition of the breathing sequence differs from at least one other repetition of the breathing sequence, for example in the number and/or type and/or sequence of breaths. In other words, the breathing sequence can be varied from repetition to repetition during ventilation. This allows the breathing sequence to be adjusted to suit changes in the patient's condition. Alternatively, the breathing sequence can be the same in each repetition.

The various repetitions of the breathing sequence can follow one another immediately, at least to some extent. The first breath in a current repetition may be immediately preceded by the last breath in an earlier repetition that immediately precedes the current repetition, and/or the last breath in a current repetition can be immediately followed by the first breath in a later repetition that immediately follows the current repetition.

For example, in at least one or every repetition, in addition to a second breath, the breathing sequence may comprise a sequence of at least two or at least four immediately consecutive first breaths, the second breath may be immediately preceded by the sequence of the first breaths.

The method can be computer-implemented, for example.

The control unit may comprise elements for data processing. The data processing elements may be implemented as hardware and/or software and/or comprise a processor. The processor can be configured to carry out the (computer-implemented) method. In addition to the processor, the control unit may comprise at least one of the following elements for data processing: a memory, a bus system for data communication between the memory and the processor, a data communication interface for wireless and/or wired data communication with peripheral devices. Alternatively, the control unit can be implemented exclusively as hardware, for example in the form of an ASIC or FPGA component.

In a second aspect, the invention provides a medical ventilator. The ventilator comprises a respiratory air connection for connecting a patient's respiratory apparatus, allowing the patient to be ventilated with breathable air. In addition, the ventilator comprises an actuator device for providing a respiratory air flow at the respiratory air connection. In addition, the ventilator comprises a control unit as described above and below. For example, the control unit may be integrated into a ventilator housing. Alternatively, an external control unit is possible.

For example, a “ventilator” can be understood to mean a machine for the invasive and/or non-invasive ventilation of the patient, and/or an anesthesia machine.

The respiratory air connection can be connected to the breathing apparatus via one or more breathing tubes and/or via a suitable patient interface such as a mask, a nasal cannula or an airway tube.

The actuator system can comprise one or more electropneumatic actuators. For example, the actuator device may comprise one or more blowers and/or one or more electrically controllable valves.

In a third aspect, the invention provides a computer program for operating a ventilator as described above and below. The computer program comprises commands that cause the control unit, such as a processor of the control unit, to carry out the following (computer-implemented) method upon execution of the computer program by the control unit, when the respiratory apparatus is connected to the respiratory air connector: generating a control signal for controlling the actuator device, so that at least one actual quantity relevant to the ventilation, which comprises a pressure and/or a volume of the respiratory air, follows a target curve between a lower limit and an upper limit during each breath, wherein in an inhalation phase in which the patient should inhale, the target curve rises from the lower limit to the upper limit and in an exhalation phase in which the patient should breathe out, it falls from the upper limit to the lower limit; (during the ventilation and/or when the actuator device is controlled:) switching the lower limit between a first setpoint and a second setpoint, which is smaller in magnitude than the first setpoint, according to a cyclically repeating breathing sequence, wherein the breathing sequence in each repetition comprises one or more first breaths, in which the at least one actual quantity is expected to fall to the first setpoint in the (respective) exhalation phase, and one or more second breaths, in which the at least one actual quantity is expected to fall to the second setpoint in the (respective) exhalation phase, wherein each second breath or each sequence of immediately consecutive second breaths is immediately preceded by a first breath or a sequence of immediately consecutive first breaths.

In a fourth aspect, the invention provides a computer-readable medium on which a computer program as described above and below is stored.

The computer-readable medium can be a volatile or non-volatile data storage device. For example, the computer-readable medium may be a hard disk, a USB (universal serial bus) storage device, a RAM (random-access memory), a ROM (read-only memory), an EPROM (erasable programmable read-only memory), an EEPROM (electrically erasable programmable read-only memory), a flash memory, or a combination of at least two of these examples. The computer-readable medium can also be a data communication network that allows program code to be downloaded (for example, over the Internet), or a cloud.

It should be noted that features of the control unit described above and below may also be features of the computer program and/or the computer-readable medium (and vice versa).

Various embodiments of the invention are described below. These embodiments are not to be understood as limiting the scope of the invention.

According to one embodiment, the exhalation phase of each first breath can be associated with a descending first portion of the target curve. In a similar manner, the exhalation phase of each second breath can be associated with a descending second portion of the target curve. The first portions and the second portions can be matched to each other in their respective duration and/or amplitude in each repetition of the breathing sequence such that a volume exhaled by the patient at each second breath is no larger than a volume exhaled by the patient at each first breath. In other words, the target curve may be defined in such a way that the patient's respiratory or tidal volume does not change significantly from one breath to another, in particular does not significantly increase at each transition from a first breath to a second breath. This ensures that the ventilation is as stress-free as possible. The term “amplitude” can be understood to mean a difference between the respective upper limit and the respective lower limit (to which the respective actual quantity should fall in the exhalation phase).

According to one embodiment, the method can further comprise: receiving an elasticity value indicating an elasticity of at least one part of the patient's respiratory apparatus and/or a resistance value indicating a flow resistance in at least one part of the patient's respiratory apparatus; determining the respective duration and/or amplitude of the first and/or second portions using the elasticity value and/or the resistance value. For example, the elasticity value can indicate a measured and/or estimated compliance of the patient's lungs and/or the resistance value can indicate a measured and/or estimated resistance of the patient's airways. This allows a simple adaptation of the respective portions of the target curve to different physiological states of the respiratory system with a view to making the ventilation process as stress-free as possible.

According to one embodiment, a product can be calculated by multiplying the elasticity value and the resistance value. The product can then be used to determine the respective duration and/or amplitude of the first and/or second portions. The product can be, for example, a time constant. In other words, the elasticity value and the resistance value can be combined into a single value by calculating their product. This can simplify subsequent calculation steps in the control unit.

According to one embodiment, the greater the magnitude of the product, the longer the respective duration of the first portions and/or the second portions may be chosen. In addition or alternatively, the greater the magnitude of the product the higher the amplitude of the first and/or second portions can be chosen. This allows a simple scaling of the duration and/or amplitude depending on the state of the respiratory apparatus.

In addition or alternatively, the elasticity value and/or the resistance value and/or the product of the elasticity value and the resistance value can be used to determine the number of second breaths in each repetition of the breathing sequence and/or to determine a ratio of the number of first breaths to the number of second breaths in each repetition of the breathing sequence.

According to one embodiment, the method can further comprise: receiving a difference value for each breath; determining a current value of the upper limit for each individual breath by adding the difference value to a current value of the lower limit. It is possible that the same difference value is received for each breath. Alternatively, at least to some extent, different difference values can be received for different breaths. For example, the difference value may have been determined taking into account a maximum ventilation pressure and/or volume with which the patient may at most be ventilated during the inhalation phase. The current lower limit value can be the first setpoint, the second setpoint, or any other setpoint, such as a third setpoint (see below), depending on the current type of breath according to the breathing sequence.

According to one embodiment, the method can further comprise: receiving an alternative difference value for each breath that immediately follows a second breath; determining a current value of the upper limit for each individual breath that immediately follows the second breath, by adding the alternative difference value instead of the difference value to a current value of the lower limit. The lower limit in the inhalation phase of the respective breath immediately following the second breath is normally set to the second (lower) setpoint according to the previous exhalation phase. This reduction can be taken into account in a suitable manner, for example compensated for, when determining the upper limit for each breath using an appropriately adjusted alternative difference value.

In other words, the difference between the lower limit and the upper limit can be varied in a targeted manner depending on the breath in relation to the inhalation and/or exhalation phase. For example, the difference value for each breath that immediately follows a second breath may be chosen greater by a certain amount than for each breath that immediately precedes a second breath, in particular in such a way that the upper limit for these two breaths is equal. Alternatively, the same difference value can be received for each breath. In this case, the upper limit may fluctuate accordingly (for example periodically).

According to one embodiment the alternative difference value can be equal to a sum obtained by adding the difference value to a difference between the first setpoint and the second setpoint. This allows the upper limit in each first breath immediately following a second breath to be the same as in each first breath immediately preceding a second breath.

According to one embodiment, a ratio of the number of first breaths to the number of second breaths in each repetition of the breathing sequence and/or per unit time, for example per minute, can be at least about two to one. The ratio may be fixed or may be varied from repetition to repetition during ventilation depending on the patient's condition. For example, the ratio of the number of first breaths to the number of second breaths per unit time may be three to one, four to one, five to one, or more than five to one. In some cases, a ratio of one-to-one may also be practical.

According to one embodiment, the exhalation phase of each second breath may last no longer than about 3 seconds, preferably no longer than about 1 second. With such a duration, experience has shown that a sufficiently effective increase in the (average) carbon dioxide elimination can be achieved without significantly increasing the tidal volume or the risk of lung collapse.

According to one embodiment the value of the second setpoint can be from about 20 to about 70 percent of the first setpoint. A ratio in this percentage range has proved to be particularly favorable in practice.

According to one embodiment the lower limit can be switched between the first setpoint, the second setpoint and a third setpoint, located between the first setpoint and the second setpoint, according to the breathing sequence. In this case the breathing sequence in at least one (or every) repetition can further comprise one or more third breaths, in which the at least one actual quantity in the (respective) exhalation phase is expected to fall to the third setpoint. Also, each first breath immediately preceding a second breath or a sequence of immediately consecutive second breaths, or each sequence of first breaths immediately preceding a second breath or a sequence of immediately consecutive second breaths, can be immediately preceded by a third breath or a sequence of immediately consecutive third breaths. The third setpoint may be, for example, a reference value, in particular in the form of a normal PEEP value, which should be used for a predominant proportion or at least for half of the breaths per repetition or per unit time. Alveolar ventilation can be further improved by first raising the lower limit from this reference value to the first setpoint before lowering it to the second setpoint in the following exhalation phase.

For example, in at least one or every repetition, in addition to a first breath and a second breath the breathing sequence may comprise a sequence of at least two or at least four immediately consecutive third breaths, wherein the second breath may be immediately preceded by the first breath and the first breath by the sequence of the third breaths.

According to one embodiment the value of the third setpoint can be from about 50 to about 80 percent of the first setpoint. A ratio in this percentage range has proved to be particularly favorable in practice.

According to one embodiment, the ventilator may further comprise a sensor device for generating measurement data relating to the ventilation. In this case, the method can further comprise receiving the measurement data in multiple consecutive time steps. The measurement data can comprise at least one of the following data types: volume data indicating a carbon dioxide volume (for example, a carbon dioxide volume per minute) exhaled by the patient; partial pressure data indicating a (for example end-tidal) carbon dioxide partial pressure in respiratory air exhaled by the patient and/or in the patient's blood; image data indicating a two- and/or three-dimensional extent of air-filled regions of the patient's lungs. The image data can preferably be generated by means of electrical impedance tomography, or EIT for short. Other invasive or non-invasive imaging methods are also possible. In addition, the method can comprise the following step: determining analysis data showing an estimated curve of a carbon dioxide elimination during the ventilation, using the measurement data from at least two time steps. For example, the analysis data can be determined in each time step from the measurement data of a current time step and at least one time step preceding the current time step.

In addition, the method can comprise the following step or steps: displaying the analysis data on a display and/or using the analysis data (and/or the measurement data) for generating the control signal and/or for adjusting the target curve (for example by changing the first setpoint and/or the second setpoint and/or the difference between the first setpoint and the second setpoint), in particular in such a way that the estimated curve of the carbon dioxide elimination approximates to a desired value range.

### BRIEF DESCRIPTION OF THE DRAWINGS

In the following, embodiments of the invention are described with reference to the accompanying drawings. Neither the description nor the drawings are to be understood as limiting the scope of the invention.

FIG. 1 shows a ventilator in accordance with one embodiment of the invention.

FIG. 2 shows a target pressure curve according to a cyclically repeating breathing sequence of first and second breaths, for use in a method which can be carried out by a control unit according to an embodiment of the invention.

FIG. 3 shows a target pressure curve according to a cyclically repeating breathing sequence of first, second and third breaths, for use in a method which can be carried out by a control unit according to an embodiment of the invention.

The drawings are purely schematic and not drawn to scale. Where the same reference signs are used in different drawings, those reference signs indicate the same or equivalent features.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

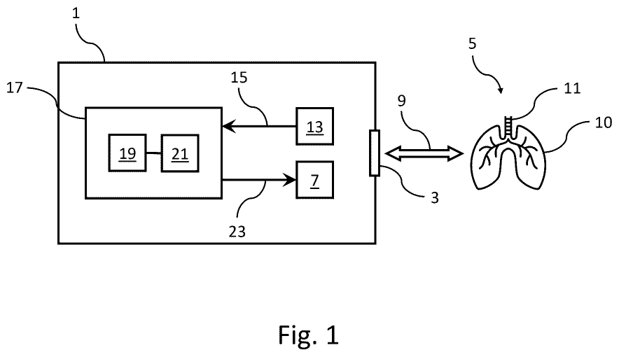

FIG. 1 shows a medical ventilator 1 for invasive and/or non-invasive ventilation of a patient. The ventilator 1 comprises a respiratory air connection 3 to which the patient's breathing apparatus 5 is connected so that respiratory air can be used to ventilate the patient, and an actuator device 7 for providing a respiratory air flow 9 at the respiratory air connection 3. For example, the respiratory air connection 3 may be connected to the patient's lungs 10 and/or airways 11 via one or more breathing tubes and a suitable patient interface such as a mask, a nasal cannula or an airway tube.

In addition, the ventilator 1 comprises a sensor device 13 for generating measurement data 15 relating to the ventilation. The sensor device 13 and the actuator device 7 can each be connected to a control unit 17. The control unit 17 can be designed to control the actuator device 7 using the measurement data 15.

The actuator device 7 may comprise one or more blowers and/or one or more electrically controllable valves.

The sensor device 13 can comprise one or more sensors, for example, at least one of the following sensors: a carbon dioxide sensor for detecting a carbon dioxide partial pressure in the respiratory air and/or in the blood of the patient; an oxygen sensor for detecting an oxygen partial pressure in the respiratory air and/or in the blood of the patient; a flow sensor for detecting a volumetric flow of the respiratory air; a pressure sensor for detecting a pressure of the respiratory air; a plurality of electrodes for measuring changes in electrical resistance in the lung tissue of the patient, for example in the context of an electrical impedance tomography procedure.

Accordingly the measurement data 15 can comprise at least one of the following data types: volumetric data indicating a volume of carbon dioxide and/or oxygen exhaled by a patient (for example, in each case as volumes per minute); partial pressure data indicating the (for example, end-tidal) carbon dioxide and/or oxygen partial pressure; image data indicating a two- and/or three-dimensional extent of air-filled regions of the patient's lungs.

The image data can, for example, encode a brightness and/or color for each image point (also called pixel) of a two- or three-dimensional matrix of pixels with respect to one or more cross-sectional planes of the lung 10. The brightness and/or color may vary as a function of a measured electrical resistance associated with the respective pixel. For example, each pixel can be assigned three values between 0 and 100 percent or between 0 and 255 (with 8 bits) for encoding the brightness of each of the colors red, green and blue. Other color spaces and/or other types of encoding are also possible.

The control unit 17 may comprise a processor 19 and a memory 21 in which a computer program for operating the ventilator 1 may be stored. The processor 19 can be configured to carry out the following method for operating the ventilator 1 by executing the computer program.

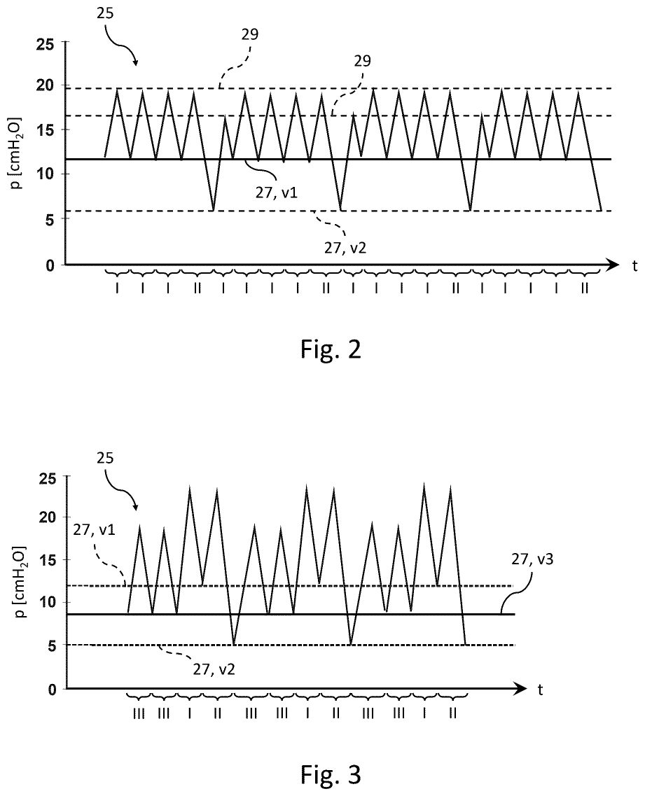

In a first step, a control signal 23 is generated for controlling the actuator device 7 so that at least one actual quantity relevant to the ventilation, which can comprise a pressure p and/or a volume of the respiratory air, follows a target curve 25 between a lower limit 27 and an upper limit 29 for each breath (see FIG. 2 and FIG. 3). In this example, the actual quantity is a pressure p of the respiratory air. In an inhalation phase, in which the patient should inhale, the target curve 25 can rise from the lower limit 27 (here a lower target pressure) to the upper limit 29 (here an upper target pressure) and in an exhalation phase, in which the patient should exhale, it can fall from the upper limit 29 to the lower limit 27.

In a second step, the lower limit 27 is switched between a first setpoint v1 (in this case a first setpoint pressure value) and a second setpoint v2 (in this case a second setpoint pressure value), which is smaller in magnitude than the first setpoint v1, according to a predefined, cyclically repeated breathing sequence. The breathing sequence in each repetition may comprise one or more first breaths I, in which the pressure p in the respective exhalation phase is expected to drop to the first setpoint v1, and one or more second breaths II, in which the pressure p in the respective exhalation phase is expected to fall to the second setpoint v2. Each second breath II or each sequence of immediately consecutive second breaths II may be immediately preceded by a first breath I or a sequence of immediately consecutive first breaths I.

The first step and the second step can be executed simultaneously or at different times.

In the example shown in FIG. 2, each second breath II is preceded by a sequence of four (or three) immediately consecutive first breaths I. Accordingly, a ratio of the number of first breaths I to the number of second breaths II in each repetition, for example per minute, is four (or three) to one. Other ratios, such as one-to-one, two-to-one, or five-to-one, are also possible. Preferably, the value of the second setpoint v2 is between 20 and 70 percent of the first setpoint v1.

As shown in FIG. 3, the breathing sequence in each repetition can further comprise one or more third breaths III, in which the pressure p in the respective exhalation phase is expected to drop to a third setpoint v3 (here a third setpoint pressure value). The size of the third setpoint v3 (here 8 cmH₂O) can be between the first setpoint v1 (here 12 cmH₂O) and the second setpoint v2 (here 5 cmH₂O). Preferably, the value of the third setpoint v3 is between 50 and 80 percent of the first setpoint v1.

Thus, it is possible that the lower limit 27 is switched between the first setpoint v1, the second setpoint v2 and the third setpoint v3 according to the breathing sequence. In this example, each first breath I that immediately precedes a second breath II is immediately preceded by a sequence of two immediately consecutive third breaths III. Accordingly, a ratio between the number of third breaths III, the number of first breaths I and the number of second breaths II in each repetition, for example per minute, is two to one to one. Other suitable ratios, such as one to one to one, are also possible, however. It is also conceivable for the ratio to be continuously varied during the ventilation depending on the patient's condition.

The third setpoint v3 may be, for example, a normal PEEP value, which should be used for a predominant proportion or at least for half of the breaths per repetition or per unit time. Alveolar ventilation can be further improved by first raising the lower limit 27 from the normal PEEP value to the first setpoint v1 before lowering it to the second setpoint v2 in the following exhalation phase.

It is possible that the exhalation phase of each first breath I is associated with a descending first portion of the target curve 25, the exhalation phase of each second breath II with a descending second portion of the target curve 25 and the exhalation phase of each third breath III with a descending third portion of the target curve 25. The respective portions of the different breath types I, II, III may be matched to each other in their respective duration and/or amplitude in each repetition of the breathing sequence such that a volume exhaled by the patient with each breath remains more or less equal over multiple breaths, in particular does not significantly increase on each transition from a first breath I to a second breath II. Thus, despite a significantly increased carbon dioxide elimination on average, the ventilation process can be made as stress-free as possible.

In order to enable an adjustment of the respective duration and/or amplitude—in particular of the second portions—to the particular condition of the patient, in an optional step an elasticity value indicating the elasticity of at least part of the respiratory apparatus 5 (for example, a compliance of the lungs 10) can be received. In addition or alternatively, a resistance value indicating a flow resistance in at least a part of the respiratory apparatus 5 (for example, a resistance of the airways 11) can be received. The elasticity value and the resistance value can each be a measured (for example by means of one or more suitable sensors of the sensor device 13) and/or estimated value.

For example, the elasticity value and the resistance value can be multiplied together. The resulting product, for example in the form of a time constant, can then be used to determine the respective duration and/or amplitude of the first and/or second portions. The greater the magnitude of the product, the longer the duration can be chosen. Similarly, the greater the magnitude of the product, the larger the amplitude can be chosen. This allows a simple scaling of the duration and/or amplitude depending on the state of the respiratory apparatus 5.

In addition or alternatively, the elasticity value and/or the resistance value and/or the product of the elasticity value and the resistance value can be used to determine the number of second breaths II in each repetition of the breathing sequence and/or to determine a ratio of the number of second breaths II to the number of first breaths I and/or to the number of third breaths III in each repetition of the breathing sequence.

In practice, a maximum duration of about 1 to 3 seconds for the exhalation phase of each second breath II has proven successful. However, other values are also possible depending on the patient's condition.

The value of the upper limit 29 can be determined, for example, by receiving a suitable difference value (here a suitable pressure differential) for each individual breath I, II or III and then adding the difference value to a current value v1, v2 or v3 of the lower limit 27 in order to obtain a current value of the upper limit 29 for the respective breath I, II or III.

The difference value can be the same for each breath. Alternatively, at least to some extent, different difference values, i.e. differing from each other in magnitude, can be received for different breaths.

One possibility, for example, is that for each breath I or III which immediately follows a second breath II, an alternative difference value (here an alternative pressure differential) is received, which deviates in magnitude from the (normal) difference value in a suitable manner, for example being greater in magnitude than the (normal) difference value. The current value of the upper limit 29 for the respective breath I or III can then be determined by adding the alternative difference value instead of the (normal) difference value to the respective current value of the lower limit 27, here v2. For example, the alternative difference value can be equal to a sum obtained by adding the (normal) difference value to a difference between the first setpoint v1 and the second setpoint v2.

The measurement data 15 can be received, for example, in multiple consecutive time steps, for example with a frequency between 1 Hz and 1000 Hz, preferably between 100 Hz and 300 Hz. From the measurement data 15 of different time steps, in a further optional step analysis data can be determined which indicate an estimated, for example time-dependent, curve of the carbon dioxide elimination during ventilation. The analysis data can then be displayed on a display, for example in the form of numerical values and/or a graph, for monitoring purposes.

In addition or alternatively, the analysis data can be used for generating the control signal 23 and/or for adjusting at least one portion of the target curve 25, for example by changing at least one of the setpoints v1, v2 and v3. It is conceivable, for example, that the target curve 25 (or at least a section of it) in a current time step is adjusted such that the estimated curve of the carbon dioxide elimination in a future time step is within a desired value range or is at least closer to the desired value range in the future time step than in the current time step.

It is well known that the functional residual capacity, FRC for short, is not constant in healthy people, but fluctuates with low cyclic frequencies of about 1 to 500 breaths, which has a corresponding effect on the gas exchange. The ventilation mode described above takes this physiological behavior into account, but accelerates such fluctuations in order to achieve additional carbon dioxide elimination during mechanical ventilation. For this purpose, repeated, short and controlled reductions of the functional residual capacity can be made every few breaths. The ventilation mode can therefore also be referred to as variable FRC ventilation. Such a ventilation mode can be used as an independent ventilation mode in a ventilator or in conjunction with other devices to actively reduce anatomical and instrumental dead spaces.

The ventilation mode can consist of an automatic cyclic FRC reduction relative to a baseline, for example by lowering the PEEP value for the duration of a single breath. Thus, the PEEP reduction is particularly short, for example, shorter than 1 second. For example, such a PEEP reduction can occur every two to four breaths. The additional FRC reduction results in additional elimination of carbon dioxide from the smaller airways and the alveolar compartment. Since the carbon dioxide of these special breaths mainly comes from the alveoli, the alveolar ventilation is intensified without dead space in the airways.

The FRC reduction should be very brief in order to avoid lung collapse. In general, the duration of the FRC reduction should be significantly lower than the time constant for an airway and/or alveolar collapse. After a series of normal breaths at a baseline PEEP, the PEEP level for a single breath should be reduced so that the FRC can decrease. This temporary reduction in FRC can be compensated for as early as during the subsequent inhalation phase. The ventilator should therefore be able to provide a sufficiently high volume flow for the inhalation to not only restore the FRC, but also provide the desired tidal volume.

For example, the ventilation mode may be configured such that the PEEP value decreases or increases by approximately 5 cmH₂O from one breath to the next, while providing a sufficiently high airflow during the inhalation phase, so that any change in the FRC can be quickly compensated and normal tidal ventilation is maintained. The pressure difference between the upper and lower target pressures should be below 15 mmHg to allow ventilation that is as gentle as possible on the lungs.

The number of FRC changes per unit time, for example per minute, and/or their amplitude, can be set manually. Alternatively, the ventilator can automatically adjust the settings.

It is possible for the ventilation mode to automatically generate a sequence of FRC changes at constant tidal volume depending on the respective difference value. In this case the total exhaled volume per minute comprises a first part corresponding to the product of tidal volume and respiratory rate, and a second part corresponding to the additionally exhaled volume due to the additional PEEP reduction. For example, a standard respiratory volume per minute of 6.75 l/min (product of a tidal volume of 450 ml and a respiratory rate of 15/min) can be specified, together with a repeated short PEEP reduction, initially from 10 cmH₂O to 5 cmH₂O and then back to 10 cmH₂O again. Assuming that such a PEEP reduction causes an additional exhalation of 200 ml at every fourth breath, an additional ventilation of approximately 1 l/min (200 ml times 5 breaths per minute) can be achieved, resulting in a total respiratory volume per minute of 7.75 l/min. In other words: The lower the ratio between normal breaths and special breaths (with lowered PEEP) and the greater the PEEP reduction, the greater is the effect of the additional carbon dioxide elimination.

With the aid of a breathing sequence, as illustrated by way of example in FIG. 3, the amplitude of the FRC change can be additionally increased and the alveolar ventilation can be improved accordingly. For example, the lower limit 27 may fluctuate in a regular manner by plus/minus 3 to 4 cmH₂O around a PEEP baseline. The PEEP baseline should be set so that the lungs do not collapse during exhalation. For example, with a PEEP base value of 8 cmH₂O and a ratio of one to one to one, the lower limit 27 could fluctuate in value cyclically as follows: 8-12-4-8-12-4-8-12-4 etc. (8-8-12-4-8-8-12-4-8-8-12-4 etc. for a ratio of two to one to one). Such a reduction in PEEP, here by 8 cmH₂O in each case, can significantly increase the carbon dioxide elimination. This allows the tidal volume to be reduced to a minimum when the FRC fluctuates.

Finally, it is also noted that terms such as “having”, “comprising”, “including”, “with” etc. do not exclude any other elements or steps, and indefinite articles such as “one” or “a/an” do not exclude a plurality.

It should also be noted that features or steps which have been described with reference to any one of the above embodiments can also be used in combination with other features or steps that have been described with reference to other embodiments from those described above.

Reference signs in the claims are not to be understood as limiting the scope of the subject-matter defined by the claims.

### LIST OF REFERENCE NUMERALS

1 ventilator
3 respiratory air connection
5 respiratory apparatus
7 actuator device
9 respiratory air flow
10 lungs
11 airways
13 sensor device
15 measurement data
17 control unit
19 processor
21 memory
23 control signal
25 target curve
27 lower limit
29 upper limit
P pressure
t time
v1 first setpoint
v2 second setpoint
v3 third setpoint
I first breath
II second breath
III third breath

What is claimed is:

1. A control unit for a medical ventilator, wherein the ventilator comprises:
a respiratory air connection for connecting a patient's respiratory apparatus, allowing the patient to be ventilated with respiratory air;
an actuator device for providing a respiratory air flow to the respiratory air connection;
and wherein the control unit is configured to carry out the following method when the respiratory apparatus is connected to the respiratory air connection:
generating a control signal for controlling the actuator device, so that at least one actual quantity relevant to a ventilation, which comprises a pressure (p) and/or a volume of respiratory air, follows a target curve between a lower limit and an upper limit during each breath (I, II, III), wherein in an inhalation phase, in which the patient should inhale, the target curve rises from the lower limit to the upper limit, and in an exhalation phase, in which the patient should exhale, the target curve falls from the upper limit to the lower limit;
switching the lower limit between a first setpoint (v1) and a second setpoint (v2), which is smaller in magnitude than the first setpoint (v1), according to a cyclically repeating breathing sequence, wherein the breathing sequence in each repetition comprises one or more first breaths (I) in which the at least one actual quantity is expected to fall to the first setpoint (v1) in the exhalation phase, and one or more second breaths (II) in which the at least one actual quantity is expected to fall to the second setpoint (v2) in the exhalation phase, wherein each second breath (II) or each sequence of immediately consecutive second breaths (II) is immediately preceded by a first breath (I) or a sequence of immediately consecutive first breaths (I).

2. The control unit of claim 1,
wherein the exhalation phase of each first breath (I) is assigned a descending first portion of the target curve and the exhalation phase of each second breath (II) is assigned a descending second portion of the target curve, wherein the first portions and the second portions are matched to each other in their respective duration and/or amplitude in each repetition of the breathing sequence, such that a volume exhaled by the patient at each second breath (II) is no larger than a volume exhaled by the patient at each first breath (I).

3. The control unit of claim 2, wherein the method further comprises:
receiving an elasticity value indicating an elasticity of at least one part of the patient's respiratory apparatus and/or a resistance value indicating a flow resistance in at least one part of the patient's respiratory apparatus;
determining the respective duration and/or amplitude of the first and/or second portions using the elasticity value and/or the resistance value.

4. The control unit of claim 3,
wherein a product is determined by multiplying the elasticity value and the resistance value and the product is used to determine the respective duration and/or amplitude.

5. The control unit of claim 4,
wherein the greater the amount of the product, the longer the duration is chosen; and/or
the greater the amount of the product, the larger the amplitude is chosen.

6. The control unit of claim 1, wherein the method further comprises:
receiving a differential value for each breath (I, II, III);
determining a current value of an upper limit for each individual breath (I, II, III) by adding a difference value to a current value (v1, v2, v3) of a lower limit.

7. The control unit of claim 6, wherein the method further comprises:
receiving an alternative difference value for each breath (I, III) that immediately follows a second breath (II);
determining a current value of an upper limit for each individual breath (I, III) that immediately follows the second breath (II), by adding the alternative difference value instead of the difference value to a current value (v2) of a lower limit.

8. The control unit of claim 7,
wherein the alternative difference value is equal to a sum obtained by adding the difference value to a difference between the first setpoint (v1) and the second setpoint (v2).

9. The control unit of claim 1,
wherein a ratio of the number of first breaths (I) to the number of second breaths (II) in each repetition of the breathing sequence is at least two to one; and/or
wherein the exhalation phase of each second breath (II) lasts no more than 3 seconds; and/or
wherein the value of the second setpoint (v2) is from about 20 to 70 percent of the first setpoint (v1).

10. The control unit of claim 9, wherein the exhalation phase of each second breath (II) lasts no more than 1 second.

11. The control unit of claim 1,
wherein the lower limit is switched between the first setpoint (v1), the second setpoint (v2) and a third setpoint (v3), located between the first setpoint (v1) and the second setpoint (v2), according to the breathing sequence, wherein the breathing sequence in at least one repetition further comprises one or more third breaths (III), in which at least one actual quantity in the exhalation phase is expected to fall to the third setpoint (v3), wherein each first breath (I) immediately preceding a second breath (II) or a sequence of immediately consecutive second breaths (II), or each sequence of first breaths (I) immediately preceding a second breath (II) or a sequence of immediately consecutive second breaths (II), is immediately preceded by a third breath (III) or a sequence of immediately consecutive third breaths (III).

12. The control unit of claim 1,
wherein the ventilator further comprises a sensor device for generating measurement data relating to the ventilation, and wherein the method further comprises:
receiving the measurement data in multiple consecutive time steps, the measurement data comprising at least one of the following data types: volumetric data indicating a carbon dioxide volume exhaled by the patient; partial pressure data indicating a carbon dioxide partial pressure in respiratory air exhaled by the patient and/or in the patient's blood; image data indicating a two- and/or three-dimensional extent of air-filled regions of the patient's lungs;
determining analysis data showing an estimated curve of carbon dioxide elimination during the ventilation, using the measurement data from at least two time steps.

13. The control unit of claim 12, wherein the method further comprises:
using the analysis data to generate the control signal and/or to adjust the target curve so that the estimated curve of carbon dioxide elimination approximates to a desired range of values.

14. A medical ventilator, wherein the ventilator comprises:
a respiratory air connection for connecting a patient's respiratory apparatus, allowing the patient to be ventilated with respiratory air;
an actuator device for providing a respiratory air flow to the respiratory air connection;
the control unit of claim 1.

15. A computer program for operating the ventilator of claim 14, wherein the computer program comprises commands which, during execution of the computer program by the control unit, cause the control unit to carry out the following method when the breathing apparatus is connected to the respiratory air connection:
generating a control signal for controlling the actuator device, so that at least one actual quantity relevant to the ventilation, which comprises a pressure (p) and/or a volume of respiratory air, follows a target curve between a lower limit and an upper limit during each breath (I, II, III), wherein in an inhalation phase, in which the patient should inhale, the target curve rises from the lower limit to the upper limit and in an exhalation phase, in which the patient should exhale, it falls from the upper limit to the lower limit;
switching the lower limit between a first setpoint (v1) and a second setpoint (v2), which is smaller in magnitude than the first setpoint (v1), according to a cyclically repeating breathing sequence, wherein the breathing sequence in each repetition comprises one or more first breaths (I) in which the at least one actual quantity is expected to fall to the first setpoint (v1) in the exhalation phase, and one or more second breaths (II) in which the at least one actual quantity is expected to fall to the second 10 setpoint (v2) in the exhalation phase, wherein each second breath (II) or each sequence of immediately consecutive second breaths (II) is immediately preceded by a first breath (I) or a sequence of immediately consecutive first breaths (I).

16. A computer-readable medium on which the computer program of claim 15 is stored.
